(19) United States
(12) Patent Application Publication
(10) Pub. No.: US 2010/0179153 A1
(43) Pub. Date: Jul. 15, 2010

(54) Bicyclic S1P Receptor Modulators

(76) Inventors: Henri Mattes, Basel (CH); Joachim Nozulak, Basel (CH); David Orain, Basel (CH); Kumlesh K. Dev, Horsham (GB)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/597,100

(22) PCT Filed: Apr. 21, 2008
(86) PCT No.: PCT/EP08/54797

§ 371 Date: Mar. 3, 2010

(30) Foreign Application Priority Data
Apr. 23, 2007 (EP) 07106753.2

(51) Int. Cl. A61K 31/502; C07D 237/32; C07D 217/24; A61K 31/47; A61P 25/00; A61P 37/00

(52) U.S. Cl. 514/248; 544/237; 546/141; 514/309

(57) ABSTRACT

The invention relates to novel heterocyclic compounds of the formula

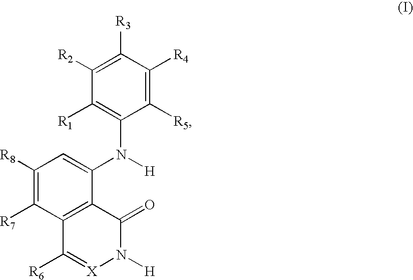

in which all of the variables are as defined in the specification, in free form or in salt form, to their preparation, b their use as medicaments and to medicaments comprising them.

The present invention relates to novel heterocyclic compounds, to their preparation, to their use as medicaments and to medicaments comprising them.

More particularly, the invention relates to a compound of the formula

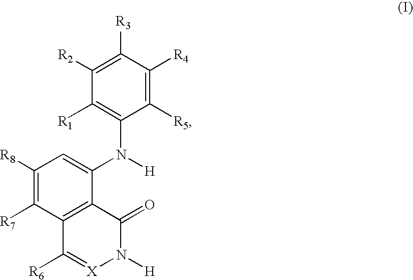

in which
R₁ and R₅ have both, in each case, identical meanings and are C₁-C₆-alkyl; C₁-C₆-alkoxy; Cl; Br; or CF₃;
R₂ and R₄ have both, in each case, identical meanings and are hydrogen; C₁-C₆-alkyl; C₁-C₆-alkoxy; F; Cl; Br; or CF₃;
R₃ is hydrogen; C₁-C₄-alkoxy; F; Cl; CF₃; or an optionally mono- or di-substituted C₁-C₈-alkyl group, the optional substituent(s) on the said alkyl group being independently selected from the group, consisting of halogen, nitro, cyano, formyl, C₁-C₄-alkylcarbonyl, hydroxy, C₁-C₄-alkoxy, formyloxy, C₁-C₄-alkylcarbonyloxy, C₁-C₄-alkoxycarbonyloxy, amino, C₁-C₄-alkylamino, formylamino, C₁-C₄-alkylcarbonylamino and C₁-C₄-alkoxycarbonylamino;
R₆ is hydrogen; an optionally mono- or di-substituted C₁-C₈-alkyl, C₂-C₄-alkenyl or C₃-C₇-cycloalkyl group, the optional substituent(s) on the said alkyl, alkenyl or cycloalkyl group being independently selected from the group, consisting of halogen, nitro, cyano, formyl, C₁-C₄-alkylcarbonyl, hydroxy, C₁-C₄-alkoxy, formyloxy, C₁-C₄-alkylcarbonyloxy, C₁-C₄alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, formylamino, C₁-C₄-alkylcarbonylamino and C₁-C₄-alkoxycarbonylamino; an optionally mono- or di-substituted heteroaryl group, the optional substituent(s) on the said heteroaryl group being independently selected from the group, consisting of halogen, nitro, cyano, formyl, C₁-C₄-alkylcarbonyl, hydroxy, C₁-C₄-alkoxy, C₁-C₄-alkyl, hydroxy-C₁-C₄-alkyl, C₁-C₄-alkoxy-C₁-C₄-alkyl, formyloxy, C₁-C₄-alkylcarbonyloxy, HO—C(═O)—, C₁-C₄-alkoxycarbonyl, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)-amino-C₁-C₄-alkyl with two identical or different C₁-C₄-alkyl moieties in the di-(C₁-C₄-alkyl)-amino moiety, pyrrolidyl-C₁-C₄-alkyl, piperidyl-C₁-C₄-alkyl, morpholinyl-C₁-C₄-alkyl, formyl-amino, C₁-C₄-alkylcarbonylamino and C₁-C₄-alkoxycarbonylamino; a heteroaryl-C₁-C₄-alkyl group, which is optionally mono- or di-substituted on the heteroaryl moiety, the optional substituent(s) on the said heteroaryl moiety being independently selected from the group, consisting of halogen, nitro, cyano, formyl, C₁-C₄-alkylcarbonyl, hydroxy, C₁-C₄-alkoxy, C₁-C₄-alkyl, hydroxy-C₁-C₄-alkyl, C₁-C₄-alkoxy-C₁-C₄-alkyl, formyloxy, C₁-C₄-alkylcarbonyloxy, HO—C(═O)—, C₁-C₄-alkoxycarbonyl, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl, formylamino, C₁-C₄-alkylcarbonylamino and C₁-C₄-alkoxycarbonylamino; an optionally mono- or di-substituted phenyl group, the optional substituent(s) on the said phenyl group being independently selected from the group, consisting of cyano, formyl, C₁-C₄-alkylcarbonyl, hydroxy, hydroxy-C₁-C₄-alkyl, C₁-C₄-alkoxy, C₁-C₄-alkoxy-C₁-C₄-alkyl, HO—C(═O)—, C₁-C₄-alkoxycarbonyl, formyloxy, C₁-C₄-alkylcarbonyloxy, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl and C₁-C₄-alkoxycarbonylamino; or an optionally mono- or di-substituted non-aromatic heterocyclyl group, the optional substituent(s) on the said heterocyclyl group being independently selected from the group, consisting of C₁-C₄-alkyl, hydroxy-C₁-C₄-alkyl, C₁-C₄-alkoxy-C₁-C₄-alkyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl, formyl, C₁-C₄-alkylcarbonyl, formyloxy, C₁-C₄-alkyl-carbonyloxy, formylamino and C₁-C₄-alkylcarbonylamino;
R₇ is hydrogen; an optionally mono- or di-substituted C₁-C₈-alkyl, C₂-C₄-alkenyl or C₃-C₇-cycloalkyl group, the optional substituent(s) on the said alkyl, alkenyl or cycloalkyl group being independently selected from the group, consisting of halogen, nitro, cyano, formyl, C₁-C₄-alkylcarbonyl, hydroxy, C₁-C₄-alkoxy, formyloxy, C₁-C₄-alkylcarbonyloxy, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, formylamino, C₁-C₄-alkylcarbonylamino and C₁-C₄-alkoxycarbonylamino; an optionally mono- or di-substituted heteroaryl group, the optional substituent(s) on the said heteroaryl group being independently selected from the group, consisting of halogen, nitro, cyano, formyl, C₁-C₄-alkylcarbonyl, hydroxy, C₁-C₄-alkoxy, C₁-C₄-alkyl, hydroxy-C₁-C₄-alkyl, C₁-C₄-alkoxy-C₁-C₄-alkyl, formyloxy, C₁-C₄-alkylcarbonyloxy, HO—C(═O)—, C₁-C₄-alkoxycarbonyl, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)-amino-C₁-C₄-alkyl with two identical or different C₁-C₄-alkyl moieties in the di-(C₁-C₄-alkyl)-amino moiety, pyrrolidyl-C₁-C₄-alkyl, piperidyl-C₁-C₄-alkyl, morpholinyl-C₁-C₄-alkyl, formylamino, C₁-C₄-alkylcarbonylamino and C₁-C₄-alkoxycarbonylamino; a heteroaryl-C₁-C₄-alkyl group, which is optionally mono- or di-substituted on the heteroaryl moiety, the optional substituent(s) on the said heteroaryl moiety being independently selected from the group, consisting of halogen, nitro, cyano, formyl, C₁-C₄-alkylcarbonyl, hydroxy, C₁-C₄-alkoxy, C₁-C₄-alkyl, hydroxy-C₁-C₄-alkyl, C₁-C₄-alkoxy-C₁-C₄-alkyl, formyloxy, C₁-C₄-alkylcarbonyloxy, HO—C(═O)—, C₁-C₄-alkoxycarbonyl, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl, formylamino, C₁-C₄-alkylcarbonylamino and C₁-C₄-alkoxycarbonylamino; an optionally mono- or di-substituted phenyl group, the optional substituent(s) on the said phenyl group being independently selected from the group, consisting of cyano, formyl, C₁-C₄-alkylcarbonyl, hydroxy, hydroxy-C₁-C₄-alkyl, C₁-C₄-alkoxy, C₁-C₄-alkoxy-C₁-C₄-alkyl, HO—C(═O)—, C₁-C₄-alkoxycarbonyl, formyloxy, C₁-C₄-alkylcarbonyloxy, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl and C₁-C₄-alkoxycarbonylamino; or an optionally mono- or di-substituted non-aromatic heterocyclyl group, the optional substituent(s) on the said heterocyclyl group being independently selected from the group, consisting of C₁-C₄-alkyl, hydroxy-C₁-C₄-alkyl, C₁-C₄-alkoxy-C₁-C₄-alkyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl, formyl, C₁-C₄-alkylcarbonyl, formyloxy, C₁-C₄-alkyl-carbonyloxy, formylamino and C₁-C₄-alkylcarbonylamino;
R₈ is hydrogen; C₁-C₄-alkyl; C₁-C₄-alkoxy; F; or Cl; and
X is CH or N,
   in free form or in salt form.

E. g. on account of one or more than one asymmetrical carbon atom, which may be present in a compound of the formula I, a corresponding compound of the formula I may exist in pure optically active form or in the form of a mixture of optical isomers, e.g. in the form of a racemic mixture. All such pure optical isomers and their mixtures, including the racemic mixtures, are part of the present invention.

A compound of the formula I may exist in free form or in salt form, e.g. a basic compound in acid addition salt form or an acidic compound in the form of a salt with a base. All such free compounds and salts are part of the present invention.

A compound of the formula I may exist in tautomeric form. All such tautomers are part of the present invention.

Halogen denotes fluorine, bromine, chlorine or iodine.

Heteroaryl is an aromatic 5- or 6-membered ring, in which 1, 2 or 3 ring atoms are hetero atoms independently selected from O, N and S, such as furyl, pyrrolyl, thienyl, thiazolyl, isothiazolyl, oxazolyl, isoxazolyl, pyridazinyl, pyrimidyl or pyridyl, and which ring can also be anellated with a phenyl ring, such as benzothiazolyl, benzoxazolyl or quinolyl.

Non-aromatic heterocyclyl is a non-aromatic 5- or 6-membered ring, in which 1, 2 or 3 ring atoms are hetero atoms independently selected from O, N and S, such as pyrrolinyl, pyrrolidyl, tetrahydrofuryl, tetrahydrothienyl, piperidyl, piperazinyl or morpholinyl.

Any non-cyclic carbon containing group or moiety with more than 1 carbon atom is straight-chain or branched.

Unless defined otherwise, carbon containing groups, moieties or molecules contain 1 to 8, preferably 1 to 6, more preferably 1 to 4, most preferably 1 or 2, carbon atoms.

In preferred embodiments, the invention relates to a compound of the formula I, in free form or in salt form, in which

(1) R₁ and R₅ have both, in each case, identical meanings and are C₁-C₆-alkyl; C₁-C₆-alkoxy; Cl; Br; or CF₃;
preferably are C₁-C₆-alkyl;
preferably are ethyl or, preferably, methyl;
(2) R₂ and R₄ have both, in each case, identical meanings and are hydrogen; C₁-C₆-alkyl; C₁-C₆-alkoxy; F; Cl; Br; or CF₃;
preferably are hydrogen;
(3) R₃ is hydrogen; C₁-C₄-alkoxy; F; Cl; CF₃; or an optionally mono- or di-substituted C₁-C₈-alkyl group, the optional substituent(s) on the said alkyl group being independently selected from the group, consisting of halogen, nitro, cyano, formyl, C₁-C₄-alkylcarbonyl, hydroxy, C₁-C₄-alkoxy, formyloxy, C₁-C₄-alkylcarbonyloxy, C₁-C₄-alkoxycarbonyloxy, amino, C₁-C₄-alkylamino, formylamino, C₁-C₄-alkylcarbonylamino and C₁-C₄-alkoxycarbonylamino;
preferably hydrogen; F; Cl; or C₁-C₈-alkyl;
preferably hydrogen; F; Cl; or C₁-C₄-alkyl;
preferably hydrogen; F; Cl; or methyl;
(4) R₆ is hydrogen; an optionally mono- or di-substituted C₁-C₈-alkyl, C₂-C₄-alkenyl or C₃-C₇-cycloalkyl group, the optional substituent(s) on the said alkyl, alkenyl or cycloalkyl group being independently selected from the group, consisting of halogen, nitro, cyano, formyl, C₁-C₄-alkylcarbonyl, hydroxy, C₁-C₄-alkoxy, formyloxy, C₁-C₄-alkylcarbonyloxy, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, formylamino, C₁-C₄-alkylcarbonylamino and C₁-C₄-alkoxycarbonylamino; an optionally mono- or di-substituted heteroaryl group, the optional substituent(s) on the said heteroaryl group being independently selected from the group, consisting of halogen, nitro, cyano, formyl, C₁-C₄-alkylcarbonyl, hydroxy, C₁-C₄-alkoxy, C₁-C₄-alkyl, hydroxy-C₁-C₄-alkyl, C₁-C₄-alkoxy-C₁-C₄-alkyl, formyloxy, C₁-C₄-alkylcarbonyloxy, HO—C(═O)—, C₁-C₄-alkoxycarbonyl, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)-amino-C₁-C₄-alkyl with two identical or different C₁-C₄-alkyl moieties in the di-(C₁-C₄-alkyl)-amino moiety, pyrrolidyl-C₁-C₄-alkyl, piperidyl-C₁-C₄-alkyl, morpholinyl-C₁-C₄-alkyl, formyl-amino, C₁-C₄-alkylcarbonylamino and C₁-C₄-alkoxycarbonylamino; a heteroaryl-C₁-C₄-alkyl group, which is optionally mono- or di-substituted on the heteroaryl moiety, the optional substituent(s) on the said heteroaryl moiety being independently selected from the group, consisting of halogen, nitro, cyano, formyl, C₁-C₄-alkylcarbonyl, hydroxy, C₁-C₄-alkoxy, C₁-C₄-alkyl, hydroxy-C₁-C₄-alkyl, C₁-C₄-alkoxy-C₁-C₄-alkyl, formyloxy, C₁-C₄-alkylcarbonyloxy, HO—C(═O)—, C₁-C₄-alkoxycarbonyl, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl, formylamino, C₁-C₄-alkylcarbonylamino and C₁-C₄-alkoxycarbonylamino; an optionally mono- or di-substituted phenyl group, the optional substituent(s) on the said phenyl group being independently selected from the group, consisting of cyano, formyl, C₁-C₄-alkylcarbonyl, hydroxy, hydroxy-C₁-Cₐ-alkyl, C₁-C₄-alkoxy, C₁-C₄-alkoxy-C₁-C₄-alkyl, HO—C(═O)—, C₁-C₄-alkoxycarbonyl, formyloxy, C₁-C₄-alkylcarbonyloxy, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl and C₁-C₄-alkoxycarbonylamino; or an optionally mono- or di-substituted non-aromatic heterocyclyl group, the optional substituent(s) on the said heterocyclyl group being independently selected from the group, consisting of C₁-C₄-alkyl, hydroxy-C₁-C₄-alkyl, C₁-C₄-alkoxy-C₁-C₄-alkyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl, formyl, C₁-C₄-alkylcarbonyl, formyloxy, C₁-C₄-alkyl-carbonyloxy, formylamino and C₁-C₄-alkylcarbonylamino;
preferably hydrogen; or an optionally mono- or di-substituted C₁-C₈-alkyl group, the optional substituent(s) on the said alkyl group being independently selected from the group, consisting of halogen, nitro, cyano, formyl, C₁-C₄-alkylcarbonyl, hydroxy, C₁-C₄-alkoxy, formyloxy, C₁-C₄-alkylcarbonyloxy, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, formylamino, C₁-C₄-alkylcarbonylamino and C₁-C₄-alkoxycarbonylamino;
preferably hydrogen; C₁-C₈-alkyl; or mono- or di-hydroxy-C₁-C₈-alkyl;
preferably hydrogen; C₁-C₈-alkyl; or mono-hydroxy-C₁-C₄-alkyl;
(5) R₇ is hydrogen; an optionally mono- or di-substituted C₁-C₈-alkyl, C₂-C₄-alkenyl or C₃-C₇-cycloalkyl group, the optional substituent(s) on the said alkyl, alkenyl or cycloalkyl group being independently selected from the group, consisting of halogen, nitro, cyano, formyl, C₁-C₄-alkylcarbonyl, hydroxy, C₁-C₄-alkoxy, formyloxy, C₁-C₄-alkylcarbonyloxy, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, formylamino, C₁-C₄-alkylcarbonylamino and C₁-C₄-alkoxycarbonylamino; an optionally mono- or di-substituted heteroaryl group, the optional substituent(s) on the said heteroaryl group being independently selected from the group, consisting of halogen, nitro, cyano, formyl, C₁-C₄-alkylcarbonyl, hydroxy, C₁-C₄-alkoxy, C₁-C₄-alkyl, hydroxy-C₁-C₄-alkyl, C₁-C₄-alkoxy-C₁-C₄-alkyl, formyloxy, C₁-C₄-alkylcarbonyloxy, HO—C(═O)—, C₁-C₄-alkoxycarbonyl, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)-amino-C₁-C₄-alkyl with two identical or different C₁-C₄-alkyl moieties in the di-(C₁-C₄-alkyl)-amino moiety, pyrrolidyl-C₁-C₄-alkyl, piperidyl-C₁-C₄-alkyl, morpholinyl-C₁-C₄-alkyl, formylamino, C₁-C₄-alkylcarbonylamino and C₁-C₄-alkoxycarbonylamino; a heteroaryl-C₁-C₄-alkyl group, which is optionally mono- or di-substituted on the heteroaryl moiety, the optional substituent(s) on the said heteroaryl moiety being independently selected from the group, consisting of halogen, nitro, cyano, formyl, C₁-C₄-alkylcarbonyl, hydroxy, C₁-C₄-alkoxy, C₁-C₄-alkyl, hydroxy-C₁-C₄-alkyl, C₁-C₄-alkoxy-C₁-C₄-alkyl, formyloxy, C₁-C₄-alkylcarbonyloxy, HO—C(═O)—, C₁-C₄-alkoxycarbonyl, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl, formylamino, C₁-C₄-alkylcarbonylamino and C₁-C₄-alkoxycarbonylamino; an optionally mono- or di-substituted phenyl group, the optional substituent(s) on the said phenyl group being independently selected from the group, consisting of cyano, formyl, C₁-C₄-alkylcarbonyl, hydroxy, hydroxy-C₁-C₄-alkyl, C₁-C₄-alkoxy, C₁-C₄-alkoxy-C₁-C₄-alkyl, HO—C(═O)—, C₁-C₄-alkoxycarbonyl, formyloxy, C₁-C₄-alkylcarbonyloxy, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl and C₁-C₄-alkoxycarbonylamino; or an optionally mono- or di-substituted non-aromatic heterocyclyl group, the optional substituent(s) on the said heterocyclyl group being independently selected from the group, consisting of C₁-C₄-alkyl, hydroxy-C₁-C₄-alkyl, C₁-C₄-alkoxy-C₁-C₄-alkyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl, formyl, C₁-C₄-alkylcarbonyl, formyloxy, C₁-C₄-alkyl-carbonyloxy, formylamino and C₁-C₄-alkylcarbonylamino; preferably hydrogen;
(6) R₈ is hydrogen; C₁-C₄-alkyl; C₁-C₄-alkoxy; F; or Cl;
preferably hydrogen;

### (7) X is CH or N;

preferably N.

The preferred embodiments (1) to (7) are preferred independently, collectively or in any combination or sub-combination.

In especially preferred embodiments, the invention relates to one or more than one of the compounds of the formula I mentioned in the Examples hereinafter, in free form or in salt form.

In a further aspect, the invention relates to a process for the preparation of a compound of the formula I, in free form or in salt form, comprising the steps of

a) for the preparation of a compound of the formula I, in which X is N, reaction of a compound of the formula

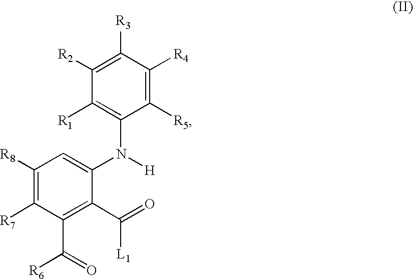

in which R₁, R₂, R₃, R₄, R₅, R₆, R₇ and R₈ are as defined for the formula I and L₁ is a leaving group, in free form or in salt form, with a compound of the formula

H₂N—NH₂ (III),

in free base form or in acid addition salt form, or
b) reaction of a compound of the formula

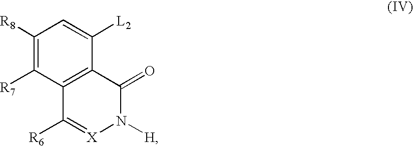

in which R₆, R₇, R₈ and X are as defined for the formula I and L₂ is a leaving group, in free form or in salt form, with a compound of the formula

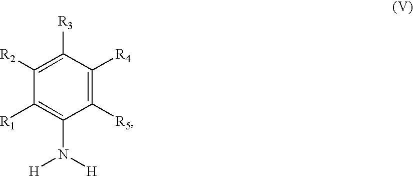

in which R₁, R₂, R₃, R₄ and R₅ are as defined for the formula I, in free form or in salt form, or
c) for the preparation of a compound of the formula I, in which X is CH, R₆ is a group of the formula (R₆ₐ)(R_{6b})(H)C (Ia) and the group of the formula Ia is selected from those groups R₆, which are bonded to the ring carbon atom carrying R₆ via a carbon atom carrying at least one hydrogen atom, intramolecular cyclisation of a compound of the formula

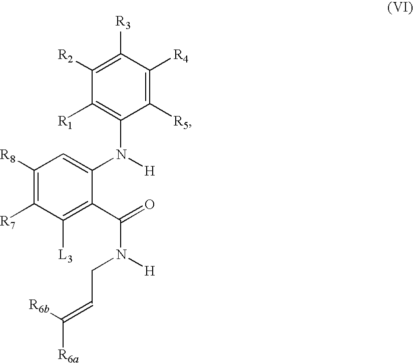

in which R₁, R₂, R₃, R₄, R₅, R₇ and R₈ are as defined for the formula I, R₆ₐ and R_{6b} are as defined for the formula Ia and L₃ is a leaving group, in free form or in salt form,
in each case optionally followed by reduction, oxidation or other functionalisation of the resulting compound and/or by cleavage of any protecting group(s) optionally present, and of recovering the so obtainable compound of the formula I in free form or in salt form.

A leaving group L₁, L₂ or L₃ is, e.g., halogen, such as F, Cl or Br, hydroxy or C₁-C₈-alkoxy, such as methoxy.

The reactions can be effected according to conventional methods, for example as described in the Examples.

The working-up of the reaction mixtures and the purification of the compounds thus obtainable may be carried out in accordance with known procedures.

Salts may be prepared from free compounds in known manner, and vice-versa.

Compounds of the formula I can also be prepared by further conventional processes, which processes are further aspects of the invention, e.g. as described in the Examples.

The starting materials of the formulae II, III, IV, V and VI are known or may be prepared according to conventional procedures starting from known compounds, for example as described in the Examples.

Compounds of the formula I, in free form or in pharmaceutically acceptable salt form, herein-after often referred to as “agents of the invention”, exhibit valuable pharmacological proper-ties, when tested in vitro or in vivo, and are, therefore, useful in medicaments.

E. g., the agents of the invention are modulators of sphingosine-1-phosphate (S1P) receptors. S1P is a bioactive sphingolipid metabolite secreted by hematopoietic cells and stored in, and released from, activated platelets. S1P acts as agonist on a family of G protein-coupled receptors (S1P receptors) to regulate, inter alia, the platelet aggregation and the cell proliferation, morphology, differentiation, chemotaxis, survival, migration and motility. Five S1P receptor subtypes have been identified: S1P, S1P₂, S1P₃, S1P₄ and S1P₅, respectively, receptors. S1P₁ receptors, e.g., regulate the T-cell trafficking, and the ligand-induced activation of S1P₁ and S1P₃ receptors, e.g., promotes the angiogenesis and chemotaxis. The agonism of S1P₂ receptors, e.g., promotes the neurite retraction and inhibits the chemotaxis. S1P₄ receptors are localized at hematopoietic cells and tissues. S1P₅ receptors are primarily neuronal receptors, e.g. expressed in oligodendrocytes and their precursors, with some expression in lymphoid tissue and NK cells and are involved, e.g., in the DNA synthesis, proliferation and migration of tumor cells and the mobilization of NK cells to inflamed organs. S1P stimulates the blood vessel growth and differentiation, but also shows cardiovascular effects, e.g. a reduced heart rate and blood pressure, that limit its therapeutic utility and are reported to be associated with its potent agonist activity on all five S1P receptor subtypes. There is, therefore, a need for further modulators of S1P receptors with, e.g., fewer therapeutic disadvantages. Surprisingly, agents of the invention have good efficacy as improved modulators of S1P receptors, which possess, e.g., desirable agonistic selectivity for one or several S1P receptor subtypes over one or several other S1P receptor subtypes. E. g., an agent of the invention can be a selective strong agonist for one S1P receptor subtype, while having modulating properties towards the other S1P receptor subtypes with, e.g., antagonistic, inverse agonistic, non-modulating or only weakly agonistic characteristics. Agents of the invention are, therefore, useful for the treatment or prevention of a condition, disease or disorder, in which the modulation of S1P receptors plays a role.

The S1P receptor modulating properties of agents of the invention can be evaluated, e.g., in a test as described hereinafter.

### Test 1: ³⁵5-GTPγS Binding Assay

The S1P receptor modulating properties of an agent of the invention are tested on the human S1P₁, S1P₂, S1P₃, S1P₄ and S1P₅, respectively, receptor subtypes. The functional receptor activation is assessed by quantifying the compound-induced ³⁵S-GTPγS binding to membrane protein prepared from transfected heterologous cells stably expressing the appropriate S1P receptor subtype. CHO cells (CHO-K1, Chinese hamster, ATCC no. CCL 61) are used [RH7777 cells (rat Morris hepatoma, ATCC no. CRL 1601) may also be used]. The membrane protein is prepared from wild-type cells and from different cell clones expressing the appropriate S1P receptor subtype. The assay technology used is SPA (scintillation proximity based assay). A DMSO solution of the test compound is serially diluted and added to SPA-bead (Amersham-Pharmacia) immobilised S1P receptor subtype expressing membrane protein (1 to 20 μg/well) in the presence of 50 mM HEPES, 100 mM NaCl, 10 mM MgCl₂, 10 μM GDP, 0.1% fat free BSA and 0.2 nM ³⁵S-GTPγS (1200 Ci/mmol) (pH 7.4). After incubation in 96 well microtiter plates at room temperature for 120 minutes, the unbound ³⁵S-GTPγS is separated by centrifugation. The luminescence of SPA beads triggered by membrane bound ³⁵S-GTPγS is quantified with a TOPcount plate reader (Packard). To evaluate the S1P receptor modulation, the stimulation (in %) compared to the baseline is calculated as the binding in the presence of the compound divided by the binding in the absence of a ligand, multiplied by 100. Dose response curves are plotted using a non-linear regression curve fitting program, and the EC₅₀ is defined as the concentration of the compound required to give 50% of its maximum stimulation. Preferably, the EC₅₀ value of an agent of the invention in this test is 10′000 nM or less. The selectivity of the compound towards the S1P receptor subtypes is determined by measuring for each of the different S1P receptor subtypes the level of ³⁵S-GTPγS binding in the presence of the compound using each of the different membrane proteins.

The results found in Test 1 are, e.g., for the agent of the invention described in Example 4 for the receptor subtype S1P₁ 2′063 nM (58% efficacy), for S1P₂ and S1P₃>10′000 nM, for S1P₄ 929 nM (74% efficacy) and for S1P₅ 117 nM (80% efficacy) and for the agent of the invention described in Example 8 for the receptor subtype S1P₁ 1′262 nM (73% efficacy), for S1P₂, S1P₃ and S1P₄>10′000 nM and for S1P₅ 27 nM (81% efficacy).

### Test 2: FLIPR Assay

CHO(CHO-K1, Chinese hamster, ATCC no. CCL 61) or RH7777 (rat Morris hepatoma, ATCC no. CRL 1601) cells, which express the desired S1P receptor subtype, are placed into black Costar plates (96 or 384 wells, 50′000 or 12′500 cells, respectively) in culture medium [CHO cells: RPMI 1640 medium (Gibco, Invitrogen Corporation), 10% FBS (heat inactivated, Gibco), 50 mg/ml gentamicin (50 mg/ml, Gibco) or 10′000 units/ml penicillin and 10 mg/ml streptomycin; RH7777 cells: DMEM (Gibco), 10% FBS (heat inactivated, Gibco), 50 mg/ml gentamicin (50 mg/ml, Gibco)] and cultured for 20 to 24 hours at 37° C. in a CO₂ incubator. In the case of the RH7777 cells, the plates are coated with poly-D-Lys. After the removal of the culture medium, the cells are incubated in HBSS medium containing 2 μM FIuo4AM (Molecular Probes, no. F-1241; 1 mg/ml stock in DMSO) and 5 mM probenicid for 1 hour at 37° C., rinsed with HBSS buffer and 2.5 mM probenicid and overlaid with the same medium (75 μl for 96 well plates, 50 μl for 384 well plates). The plates are transferred to the FLIPR. After measuring the baseline for 40 seconds, the test compound in HBSS is added, and the fluorescence is measured at intervals of 2 seconds for 3 to 5 minutes. To obtain high quality signals, the CHO cells expressing an S1P receptor subtype are pre-incubated with 10 μM ATP 20 to 30 minutes prior to the addition of the test compound. The cells can also be pre-treated for 5 hours with 50 ng/ml pertussis toxin (Sigma, no. P2980). 2-Aminoethoxydiphenyl borate (Calbiochem, no. 100065), a blocker of the release from the endoplasmic reticulum, is added (50 or 150 μM) directly to the cell medium 20 to 40 minutes prior to the measurements. The calculation of the EC₅₀ is performed using a non-linear regression curve fitting program, e.g. as provided in the Origin 7 RS2 software package (Origin LabCorporation).

### Test 3: Multiple Sclerosis Model

As a multiple sclerosis model a rodent experimental autoimmune encephalomyelitis (EAE) model may be used, e.g., the SJL/J mouse model of chronic progressive EAE. On day 0, female SJL/J mice are immunized by subcutaneous flank injection with 200 ml of inoculum containing 500 mg of bovine myelin basic protein (MBP) emulsified in complete Freund's adjuvant. On day 9, the mice are boosted by a second MBP injection and an additional intra-venous adjuvant injection consisting of 200 ng of B. pertussis toxin. A final B. pertussis injection is given on day 11. Most of the MBP-immunized mice exhibit a severe bout of EAE by day 21. This is followed by a recovery phase starting around day 25, during which time the mice remain symptom-free for about 20 days. By days 45 to 47, approximately 50% of the mice enter the progressive phase of the disease. The treatment with the test compound starts on day 21, when the disease is fully established, and continues until day 70. Recombinant mouse interferon beta (Calbiochem/Biosciences) is dissolved in saline and given by intraperitoneal injection 3 times a week. The test compound is administered by gavage 5 times a week. The mice in the vehicle control group are MBP-immunized and treated with water. Each experimental group consists of 10 mice, which are examined daily for clinical EAE symptoms using a scale ranging from 0 to 3. The disease incidence and the day of EAE onset are also recorded. A disease-related mortality, which occurs after the start of the treatment, is recorded with the maximum score of 3. In this model, a beneficial effect can be seen, when the test compound is administered at a dose of from about 1 to about 100 mg/kg.

Due to their S1P receptor modulating activities, agents of the invention are useful, e.g., in the treatment or prevention of a variety of psychiatric, psychotic, neurological, autoimmune, immunoregulatory or inflammatory conditions, disorders or diseases, in which the modulation of S1P receptors plays a role, in transplantation, e.g. for the inhibition of acute or chronic graft rejection, or as part of chemotherapeutic regimens for the treatment of cancers or tumors, e.g. of gliomas, lymphomas or leukemias.

The said psychiatric, psychotic or neurological conditions, disorders or diseases include, e.g., anxiety disorders, such as panic disorder with or without agoraphobia, agoraphobia without history of panic disorder, animal or other specific phobias, including social phobias, social anxiety disorder, anxiety, obsessive-compulsive disorder, stress disorders, including post-traumatic or acute stress disorder, or generalized or substance-induced anxiety disorders; neuroses; seizures; epilepsy, especially partial seizures, simple, complex or partial seizures evolving to secondarily generalized seizures or generalized seizures [absence (typical or atypical), myoclonic, clonic, tonic, tonic-clonic or atonic seizures]; convulsions; migraine; affective disorders, including depressive or bipolar disorders, e.g. single-episode or recurrent major depressive disorder, major depression, dysthymic disorder, dysthymia, depressive disorder NOS, bipolar I or bipolar II manic disorder or cyclothymic disorder; psycho-tic disorders, including schizophrenia; neurodegeneration arising from cerebral ischemia; acute, traumatic or chronic degenerative and/or demyelinating processes of the nervous system, such as Parkinson's disease, Down's syndrome, senile dementia, cognitive disorders, Alzheimer's disease, Huntington's chorea, amyotrophic lateral sclerosis, neuromyelitis optica, acute disseminated encephalomyelitis, allergic encephalomyelitis, Marchiafava-Bignami disease, progressive multifocal leukoencephalopathy, post-infectious encephalitis, central pontine myelinolysis, adrenoleukodystrophy, Krabbe's disease, metachromatic leuko-dystrophy, Alexander's disease, Canavan disease, Cockayne's syndrome, Pelizaeus-Merz-bacher's disease, Hurler's disease, Lowe's syndrome, spinal cord injury, transverse myelitis, Guillain-Barr syndrome, phenylketonuria, Refsum's disease, Charcot-Marie-Tooth disease, Gaucher disease, multiple sclerosis, fragile X syndrome or focal demyelinating disease; attention disorders, e.g. attention deficit hyperactivity disorder; Tourette's syndrome; speech disorders, including stuttering; disorders of the circadian rhythm, e.g. in subjects suffering from the effects of jet lag or shift work; pain or nociception; itch; emesis, including acute, delayed or anticipatory emesis, such as emesis induced by chemotherapy or radiation, motion sickness, or post-operative nausea or vomiting; eating disorders, including anorexia nervosa or bulimia nervosa; premenstrual syndrome; muscle spasm or spasticity, e.g. in paraplegic patients; hearing disorders, e.g. tinnitus or age-related hearing impairment; urinary incontinence; or substance-related disorders, including substance abuse or dependency, including substance, such as alcohol, withdrawal disorders. Agents of the invention may also be useful in enhancing cognition, e.g. in subjects suffering from dementing conditions, such as Alzheimer's disease; or as pre-medication prior to anaesthesia or minor procedures, such as endoscopy, including gastric endoscopy.

The said autoimmune, immunoregulatory or inflammatory conditions, disorders or diseases include, e.g., sarcoidosis, fibroid lung disease, idiopathic interstitial pneumonia, obstructive airways diseases, including, e.g., asthma, intrinsic asthma, extrinsic asthma, dust asthma, chronic asthma, inveterate asthma, late asthma, airways hyperreponsiveness, bronchitis, bronchial asthma or infantile asthma, allergic rheumatoid arthritis, systemic lupus erythematosus, nephrotic syndrome lupus, Hashimoto's thyroiditis, myasthenia gravis, type I diabetes mellitus and complications associated therewith, type II adult onset diabetes mellitus, uveitis, nephrotic syndrome, steroid-dependent nephrosis, steroid-resistant nephrosis, palmoplantar pustulosis, glomerulonephritis, psoriasis, psoriatic arthritis, atopic eczema, atopic dermatitis, contact dermatitis or other eczematous dermatitises, seborrheic dermatitis, lichen planus, pemphigus, bullous pemphigoid, epidermolysis bullosa, urticaria, angioedema, vasculitides, erythemas, cutaneous eosinophilia, acne, alopecia greata, eosinophilic fasciitis, atherosclerosis, conjunctivitis, keratoconjunctivitis, keratitis, vernal conjunctivitis, uveitis associated with Behcet's disease, herpetic keratitis, conical cornea, dystorphia epithelialis corneae, kerato-leukoma, ocular pemphigus, Mooren's ulcer, scleritis, Graves' opthalmopathy, severe intraocular inflammation, inflammations of the mucosa or blood vessels, such as leukotriene B4-mediated diseases, gastric ulcer, vascular damage caused by ischemic diseases or thrombosis, ischemic bowel disease, inflammatory bowel disease, Crohn's disease, ulcerative colitis, necrotizing enterocolitis, renal diseases, such as interstitial nephritis, Goodpasture's syndrome, hemolytic uremic syndrome or diabetic nephropathy, nervous diseases selected from multiple myositis, Meniere's disease and radiculopathy, collagen diseases, scleroderderma, Wegener's granuloma, Sjogren's syndrome, chronic autoimmune liver diseases, e.g. autoimmune hepatitis, primary biliary cirrhosis or sclerosing cholangitis, partial liver resection, acute liver necrosis, e.g. caused by toxins, viral hepatitis, shock or anoxia, B-virus hepatitis, non-A/non-B hepatitis and cirrhosis, fulminant hepatitis, pustular psoriasis, Behcet's disease, active chronic hepatitis, Evans syndrome, pollinosis, idiopathic hypoparathyroidism, Addison's disease, autoimmune atrophic gastritis, lupoid hepatitis, tubulointerstitial nephritis, membranous nephritis or rheumatic fever.

For the above-mentioned indications, the appropriate dosage will vary depending on, e.g., the compound employed, the host, the mode of administration, the nature and severity of the condition, disorder or disease or the effect desired. In general, satisfactory results in animals are indicated to be obtained at a daily dosage of from about 0.1 to about 100, preferably from about 1 to about 50, mg/kg of animal body weight. In larger mammals, for example humans, an indicated daily dosage is in the range of from about 0.5 to about 2000, preferably from about 2 to about 200, mg of an agent of the invention conveniently administered, for example, in divided doses up to four times a day or in sustained release form.

An agent of the invention may be administered by any conventional route, in particular enterally, preferably orally, e.g. in the form of a tablet or capsule, or parenterally, e.g. in the form of an injectable solution or suspension, topically, e.g. in the form of a lotion, gel, ointment or cream, or in the form of a nasal spray or a suppository.

In accordance with the foregoing, in a further aspect, the invention relates to an agent of the invention for use as a medicament, e.g. for the treatment or prevention of a condition, disease or disorder, in which the modulation of S1P receptors plays a role.

In a further aspect, the invention relates to the use of an agent of the invention as active ingredient in a medicament, e.g. for the treatment or prevention of a condition, disease or disorder, in which the modulation of S1P receptors plays a role.

In a further aspect, the invention relates to a pharmaceutical composition comprising an agent of the invention as active ingredient in association with at least one pharmaceutical carrier or diluent. Such a composition may be manufactured in conventional manner, e.g. by mixing its components. Unit dosage forms contain, e.g., from about 0.1 to about 1000, preferably from about 1 to about 500, mg of an agent of the invention.

An agent of the invention can be administered as sole active ingredient or as a combination with at least one other active ingredient pharmaceutically effective, e.g., in the treatment or prevention of a psychiatric, psychotic, neurological, autoimmune, immunoregulatory or inflammatory condition, disorder or disease, in which the modulation of S1P receptors plays a role, mentioned hereinabove, in transplantation, e.g. in the inhibition of acute or chronic graft rejection, or as part of chemotherapeutic regimens for the treatment of cancers or tumors, e.g. of gliomas, lymphomas or leukemias. Such a pharmaceutical combination may be in the form of a unit dosage form, whereby each unit dosage will comprise a predetermined amount of the at least two active components in admixture with at least one pharmaceutical carrier or diluent. Alternatively, the combination may be in the form of a package containing the at least two active components separately, e.g. a pack or dispenser-device adapted for the concomitant or separate administration of the two active components, wherein these active components are separately arranged. In a further aspect, the invention relates to such pharmaceutical combinations.

For example, an agent of the invention may be used in combination with a calcineurin inhibitor, e.g. a cyclosporine, an ascomycin or an immunosuppressive analogue or derivative thereof, e.g. cyclosporin A, ISA-Tx247, FK-506, ABT-281 or ASM-981; an mTOR inhibitor, e.g. rapamycin, 40-O-(2-hydroxyethyl)-rapamycin, CCI779, ABT578 or a rapalogue, e.g. AP23464, AP23573, AP23675, AP23841, TAFA-93, biolimus 7 or biolimus 9; a corticosteroid; cyclophosphamide; azathioprene; methotrexate; an S1P receptor modulator, e.g. FTY720 or an analogue thereof; leflunomide or an analogue thereof; mizoribine; mycophenolic acid or a salt, e.g. the sodium salt, thereof; mycophenolate mofetil; 15-deoxyspergualine or an analogue thereof; a PKC inhibitor, e.g. as disclosed in WO-02/38561 or WO-03/82859; an immunosuppressive monoclonal antibody, e.g. against a leukocyte receptor, e.g. MHC, CD2, CD3, CD4, CD7, CD 11a/CD18, CD25, CD 27, CD40, CD45, CD58, CD 137, CD150 (SLAM), B7, ICOS, OX40, 4-1 BB or a ligand thereof, e.g. CD154; or another immunomodulating compound, e.g. a recombinant binding molecule having at least a portion of the extracellular domain of CTLA4 or a mutant thereof, e.g. joined to a non-CTLA4 protein sequence, e.g. CTLA4Ig (ATCC 68629) or a mutant thereof, e.g. LEA29Y, or another adhesion molecule inhibitor, e.g. a monoclonal antibody or a low molecular weight inhibitor, e.g. an LFA-1 antagonist, selectin antagonist or VLA-4 antagonist.

In a further aspect, the invention relates to the use of an agent of the invention for the manufacture of a medicament for the treatment or prevention of a condition, disease or disorder, in which the modulation of S1P receptors plays a role.

In a further aspect, the invention relates to a method for the treatment or prevention of a condition, disease or disorder, in which the modulation of S1P receptors plays a role, in a subject in need of such treatment, which comprises administering to such subject a therapeutically effective amount of an agent of the invention.

The following Examples illustrate the invention, but do not limit it.

### EXAMPLES

### Abbreviations

| | |
|---|---|
| DCM | dichloromethane |
| DMF | N,N-dimethylformamide |
| ESI | electrospray ionization |
| EtOH | ethanol |
| h | hour(s) |
| HATU | N-[(dimethylamino)-1H-1,2,3-triazolo[4,5-b]pyridin-1- |
| | ylmethylene]-N-methylmethanaminium hexafluorophosphate |
| | N-oxide |
| HPLC | high performance liquid chromatography |
| min | minute(s) |
| MS | mass spectrometry |
| Pd(OAc)₂ | palladium acetate |
| rt | retention time |
| TFA | trifluoroacetic acid |
| THF | tetrahydrofuran |

### General HPLC Information

| | |
|---|---|
| System: | Gilson 331 pumps coupled to Gilson UV/VIS 152 |
| | detector and Finnigan AQA mass spectrometer |
| | (ESI); 50 μL loop injection valve |
| Column dimensions: | 50 × 4.6 mm |
| Column type: | Waters XTerra MS C18 3.5 μm |
| Eluent: | A) Water + 0.05 vol.-% TFA |
| | B) Acetonitrile + 0.05 vol.-% TFA |
| Gradient: | From 5% to 90% B |

### Example 1

### 4-Methyl-8-(2,4,6-trimethyl-phenylamino)-2H-phthalazin-1-one

### a) 2-Bromo-6-(2,4,6-trimethyl-phenylamino)-benzoic Acid

To a solution of diisopropylamine (5.45 mL, 38.4 mmol) in THF (15 mL) is added at 0° C. n-butyllithium (24 mL, 1.6M in hexane). The mixture is stirred for 15 min at 0° C. and then cooled to −78° C. 2,4,6-trimethylaniline (3.77 mL, 26.85 mmol) is added at this temperature. After stirring for 10 min, 2-bromo-6-fluoro-benzoic acid (2.8 g, 12.78 mmol) in THF (10 mL) is added at −78° C. The reaction mixture is allowed to reach room temperature and is stirred overnight, concentrated, acidified to pH 1 with 10% aqueous HCl and extracted with ethyl acetate. The organic phase is dried over Na₂SO₄ and evaporated in vacuo. The residue is triturated with hexane, and the solid is filtered off and dried in vacuo to yield 2-bromo-6-(2,4,6-trimethyl-phenylamino)-benzoic acid as a brownish solid.

ESI-MS: 334.3 [M+H]⁺; rt=6.01 min.

### b) 2-Bromo-6-(2,4,6-trimethyl-phenylamino)-benzoic Acid Methyl Ester

Trimethylsilyldiazomethane (26.0 mL, 52 mmol) is added dropwise at 0° C. to a solution of 2-bromo-6-(2,4,6-trimethyl-phenylamino)-benzoic acid (22.0 g, 43.4 mmol) in methanol (25 mL) and THF (83 mL). The mixture is stirred at 25° C. for 18 h. Acetic acid (3.9 mL) is then added, and the mixture is diluted with ethyl acetate, washed with water, aqueous sodium bicarbonate and brine, dried over Na₂SO₄ and evaporated. The residue is purified by flash chromatography using hexane to hexane/ethyl acetate 8:2 as eluent to yield 2-bromo-6-(2,4,6-trimethyl-phenylamino)-benzoic acid methyl ester as a yellow oil.

ESI-MS: 348.1 [M+H]⁺; rt=6.81 min.

### c) 2-Acetyl-6-(2,4,6-trimethyl-phenylamino)-benzoic Acid Methyl Ester

2-Bromo-6-(2,4,6-trimethyl-phenylamino)-benzoic acid methyl ester (14.0 g, 40.2 mmol), tributyl(1-ethoxyvinl)tin (20.4 mL, 60.4 mmol) and tetrakistriphenylphosphinepalladium (2.3 g, 2.0 mmol) are added to dioxane (190 mL) in an oven-dried flask. The flask is closed with a septum, and the mixture is stirred overnight at 85° C., then diluted with ethyl acetate, washed with saturated bicarbonate and brine, dried over Na₂SO₄ and evaporated. The residue is dissolved in THF (200 mL). 1N HCl (60 mL) is added, and the mixture is stirred for 18 h. The solvent is removed in vacuo, and the crude product is purified by flash chromatography using hexane to hexane/ethyl acetate 8:2 as eluent to yield a yellow residue. Ethyl acetate is added. The yellow solid is filtered off and dried to yield 2-acetyl-6-(2,4,6-trimethyl-phenyl-amino)-benzoic acid methyl ester as an orange oil.

ESI-MS: 312.2 [M+H]⁺; rt=6.31 min.

### d) 4-Methyl-8-(2,4,6-trimethyl-phenylamino)-2H-phthalazin-1-one

Hydrazine monohydrate (1.87 mL, 38.5 mmol) is added to a solution of 2-acetyl-6-(2,4,6-trimethyl-phenylamino)-benzoic acid methyl ester (6.0 g, 19.3 mmol) in ethanol (110 mL). The mixture is stirred at 95° C. for 18 h and then cooled to room temperature. The precipitated solid is filtered off, dried and recrystallized from EtOH to yield 4-methyl-8-(2,4,6-trimethyl-phenylamino)-2H-phthalazin-1-one as a beige solid.

ESI-MS: 294.2 [M+H]⁺; rt=5.92 min.

### Example 2

### 4-Methyl-8-(2,4,6-trimethyl-phenylamino)-2H-isoquinolin-1-one

### a) N-Allyl-2-bromo-6-(2,4,6-trimethyl-phenylamino)-benzamide

2-Bromo-6-(2,4,6-trimethyl-phenylamino)-benzoic acid (0.30 g, 0.90 mol) is dissolved in DMF (9 mL). HATU (0.614 g, 1.61 mmol) is added, and the brown mixture is stirred at room temperature for 3 days. Allylamine (0.69 mL, 9 mmol) is then added, and the mixture is stirred at room temperature for 1 h. Ethyl acetate and 2N NaOH are added. The organic layer is separated, washed with water, dried over Na₂SO₄ and concentrated. The residue is purified by flash chromatography using hexane to hexane/ethyl acetate 6:4 as eluent to yield an orange crude product. Hexane is added, and the precipitate is filtered off, washed with hexane and dried to yield N-allyl-2-bromo-6-(2,4,6-trimethyl-phenylamino)-benzamide as a white solid.

ESI-MS: 373.3 [M+H]⁺; rt=6.16 min.

### b) 4-Methyl-8-(2,4,6-trimethyl-phenylamino)-2H-isoquinolin-1-one

N-Allyl-2-bromo-6-(2,4,6-trimethyl-phenylamino)-benzamide (0.074 g, 0.20 mmol), Pd(OAc)₂ (0.0023 g, 0.01 mmol), tricyclohexylphosphine (0.0057 g, 0.02 mmol) and N,N-dicyclo-hexylmethylamine (0.17 mL, 0.85 mmol) are placed into a dry flask. Dimethylacetamide (2 mL) is added, and the yellow mixture is stirred overnight at 80° C. under nitrogen. The solvent is evaporated, and the residue is purified by flash chromatography using hexane to hexane/ethyl acetate 6:4 as eluent to yield a yellow crude product. Hexane is added, and the precipitate is filtered off, washed with hexane and dried to yield 4-methyl-8-(2,4,6-trimethyl-phenyl-amino)-2H-isoquinolin-1-one as a pale yellow solid.

ESI-MS: 293.3 [M+H]⁺; rt=6.41 min.

### Example 3

### 8-(2,6-Dimethyl-phenylamino)-2H-isoquinolin-1-one

A mixture of 2,6-dimethylaniline (681 μL, 5.18 mmol), 8-bromo-2H-isoquinolin-1-one (0.20 g, 0.89 mmol), K₂CO₃ (0.136 g, 0.98 mmol) and Cu (0.68 mg, 0.01 mmol) is stirred at 135° C. for 18 h, acidified to pH 0 with 2N HCl and stirred for 15 min while cooling to room temperature. The solid is filtered off, washed with 0.5N HCl and dried to yield a brown crude product, which is purified by flash-chromatography using DCM to DCM/methanol 90:10 as eluent to yield 8-(2,6-dimethyl-phenylamino)-2H-isoquinolin-1-one as an off-white solid.

ESI-MS: 265.3 [M+H]⁺; rt=5.78 min.

### Examples 4 to 10

The compounds of Examples 4 to 10 can be prepared in a manner analogous to those described hereinbefore.

| | | ESI-MS | Retention |
|---|---|---|---|
| Example | Name | [M + H]⁺ | Time/min |
| 4 | 8-(4-Fluoro-2,6-dimethyl-phenylamino)-4-methyl-2H- | 298.3 | 5.55 |
| | phthalazin-1-one | | |
| 5 | 4-Ethyl-8-(2,4,6-trimethyl-phenylamino)-2H- | 308.4 | 6.79 |
| | phthalazin-1-one | | |
| 6 | 4-Isopropyl-8-(2,4,6-trimethyl-phenylamino)-2H- | 322.4 | 6.93 |
| | phthalazin-1-one | | |
| 7 | 4-(2-Hydroxy-ethyl)-8-(2,4,6-trimethyl-phenylamino)- | 324.4 | 5.40 |
| | 2H-phthalazin-1-one | | |
| 8 | 8-(2,6-Diethyl-4-fluoro-phenylamino)-4-methyl-2H- | 326.3 | 6.22 |
| | phthalazin-1-one | | |
| 9 | 8-(4-Chloro-2,6-dimethyl-phenylamino)-4-methyl-2H- | 314.1 | 6.03 |
| | phthalazin-1-one | | |
| 10 | 4-Ethyl-8-(4-fluoro-2,6-dimethyl-phenylamino)-2H- | 312.2 | 6.03 |
| | phthalazin-1-one | | |

1. A compound of the formula 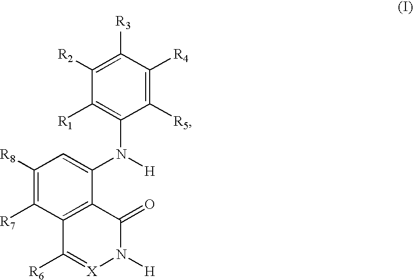
in which
R₁ and R₅ have both, in each case, identical meanings and are C₁-C₆-alkyl; C₁-C₆-alkoxy; Cl; Br; or CF₃;
R₂ and R₄ have both, in each case, identical meanings and are hydrogen; C₁-C₆-alkyl; C₁-C₆-alkoxy; F; Cl; Br; or CF₃;
R₃ is hydrogen; C₁-C₄-alkoxy; F; Cl; CF₃; or an optionally mono- or di-substituted C₁-C₈-alkyl group, the optional substituent(s) on the said alkyl group being independently selected from the group, consisting of halogen, nitro, cyano, formyl, C₁-C₄-alkylcarbonyl, hydroxy, C₁-C₄-alkoxy, formyloxy, C₁-C₄-alkylcarbonyloxy, C₁-C₄-alkoxycarbonyloxy, amino, C₁-C₄-alkylamino, formylamino, C₁-C₄-alkylcarbonylamino and C₁-C₄-alkoxycarbonylamino;
R₆ is hydrogen; an optionally mono- or di-substituted C₁-C₈-alkyl, C₂-C₄-alkenyl or C₃-C₇-cycloalkyl group, the optional substituent(s) on the said alkyl, alkenyl or cycloalkyl group being independently selected from the group, consisting of halogen, nitro, cyano, formyl, C₁-C₄-alkylcarbonyl, hydroxy, C₁-C₄-alkoxy, formyloxy, C₁-C₄-alkylcarbonyloxy, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, formylamino, C₁-C₄-alkylcarbonylamino and C₁-C₄-alkoxycarbonylamino; an optionally mono- or di-substituted heteroaryl group, the optional substituent(s) on the said heteroaryl group being independently selected from the group, consisting of halogen, nitro, cyano, formyl, C₁-C₄-alkylcarbonyl, hydroxy, C₁-C₄-alkoxy, C₁-C₄-alkyl, hydroxy-C₁-C₄-alkyl, C₁-C₄-alkoxy-C₁-C₄-alkyl, formyloxy, C₁-C₄-alkylcarbonyloxy, HO—C(═O)—, C₁-C₄-alkoxycarbonyl, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)-amino-C₁-C₄-alkyl with two identical or different C₁-C₄-alkyl moieties in the di-(C₁-C₄-alkyl)-amino moiety, pyrrolidyl-C₁-C₄-alkyl, piperidyl-C₁-C₄-alkyl, morpholinyl-C₁-C₄-alkyl, formylamino, C₁-C₄-alkylcarbonylamino and C₁-C₄-alkoxycarbonylamino; a heteroaryl-C₁-C₄-alkyl group, which is optionally mono- or di-substituted on the heteroaryl moiety, the optional substituent(s) on the said heteroaryl moiety being independently selected from the group, consisting of halogen, nitro, cyano, formyl, C₁-C₄-alkylcarbonyl, hydroxy, C₁-C₄-alkoxy, C₁-C₄-alkyl, hydroxy-C₁-C₄-alkyl, C₁-C₄-alkoxy-C₁-C₄-alkyl, formyloxy, C₁-C₄-alkylcarbonyloxy, HO—C(═O)—, C₁-C₄-alkoxycarbonyl, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl, formylamino, C₁-C₄-alkylcarbonylamino and C₁-C₄-alkoxycarbonylamino; an optionally mono- or di-substituted phenyl group, the optional substituent(s) on the said phenyl group being independently selected from the group, consisting of cyano, formyl, C₁-C₄-alkylcarbonyl, hydroxy, hydroxy-C₁-C₄-alkyl, C₁-C₄-alkoxy, C₁-C₄-alkoxy-C₁-C₄-alkyl, HO—C(═O)—, C₁-C₄-alkoxycarbonyl, formyloxy, C₁-C₄-alkylcarbonyloxy, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl and C₁-C₄-alkoxycarbonylamino; or an optionally mono- or di-substituted non-aromatic heterocyclyl group, the optional substituent(s) on the said heterocyclyl group being independently selected from the group, consisting of C₁-C₄-alkyl, hydroxy-C₁-C₄-alkyl, C₁-C₄-alkoxy-C₁-C₄-alkyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl, formyl, C₁-C₄-alkylcarbonyl, formyloxy, C₁-C₄-alkyl-carbonyloxy, formylamino and C₁-C₄-alkylcarbonylamino;
R₇ is hydrogen; an optionally mono- or di-substituted C₁-C₈-alkyl, C₂-C₄-alkenyl or C₃-C₇-cycloalkyl group, the optional substituent(s) on the said alkyl, alkenyl or cycloalkyl group being independently selected from the group, consisting of halogen, nitro, cyano, formyl, C₁-C₄-alkylcarbonyl, hydroxy, C₁-C₄-alkoxy, formyloxy, C₁-C₄-alkylcarbonyloxy, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, formylamino, C₁-C₄-alkylcarbonylamino and C₁-C₄-alkoxycarbonylamino; an optionally mono- or di-substituted heteroaryl group, the optional substituent(s) on the said heteroaryl group being independently selected from the group, consisting of halogen, nitro, cyano, formyl, C₁-C₄-alkylcarbonyl, hydroxy, C₁-C₄-alkoxy, C₁-C₄-alkyl, hydroxy-C₁-C₄-alkyl, C₁-C₄-alkoxy-C₁-C₄-alkyl, formyloxy, C₁-C₄-alkylcarbonyloxy, HO—C(═O)—, C₁-C₄-alkoxycarbonyl, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)-amino-C₁-C₄-alkyl with two identical or different C₁-C₄-alkyl moieties in the di-(C₁-C₄-alkyl)-amino moiety, pyrrolidyl-C₁-C₄-alkyl, piperidyl-C₁-C₄-alkyl, morpholinyl-C₁-C₄-alkyl, formylamino, C₁-C₄-alkylcarbonylamino and C₁-C₄-alkoxycarbonylamino; a heteroaryl-C₁-C₄-alkyl group, which is optionally mono- or di-substituted on the heteroaryl moiety, the optional substituent(s) on the said heteroaryl moiety being independently selected from the group, consisting of halogen, nitro, cyano, formyl, C₁-C₄-alkylcarbonyl, hydroxy, C₁-C₄-alkoxy, C₁-C₄-alkyl, hydroxy-C₁-C₄-alkyl, C₁-C₄-alkoxy-C₁-C₄-alkyl, formyloxy, C₁-C₄-alkylcarbonyloxy, HO—C(═O)—, C₁-C₄-alkoxycarbonyl, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl, formylamino, C₁-C₄-alkylcarbonylamino and C₁-C₄-alkoxycarbonylamino; an optionally mono- or di-substituted phenyl group, the optional substituent(s) on the said phenyl group being independently selected from the group, consisting of cyano, formyl, C₁-C₄-alkylcarbonyl, hydroxy, hydroxy-C₁-C₄-alkyl, C₁-C₄-alkoxy, C₁-C₄-alkoxy-C₁-C₄-alkyl, HO—C(═O)—, C₁-C₄-alkoxycarbonyl, formyloxy, C₁-C₄-alkylcarbonyloxy, C₁-C₄-alkoxycarbonyloxy, amino, C₁-C₄-alkylamino, di-(C₁-C₄-alkyl)amino with two identical or different C₁-C₄-alkyl moieties, pyrrolidyl, piperidyl, morpholinyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl and C₁-C₄alkoxycarbonylamino; or an optionally mono- or di-substituted non-aromatic heterocyclyl group, the optional substituent(s) on the said heterocyclyl group being independently selected from the group, consisting of C₁-C₄-alkyl, hydroxy-C₁-C₄-alkyl, C₁-C₄-alkoxy-C₁-C₄-alkyl, amino-C₁-C₄-alkyl, C₁-C₄-alkylamino-C₁-C₄-alkyl, di-(C₁-C₄-alkyl)amino-C₁-C₄-alkyl with two identical or different C₁-C₄-alkyl moieties in the di-(C₁-C₄-alkyl)amino moiety, pyrrolidyl-C₁-C₄-alkyl, piperidyl-C₁-C₄-alkyl, morpholinyl-C₁-C₄-alkyl, formyl, C₁-C₄-alkylcarbonyl, formyloxy, C₁-C₄-alkyl-carbonyloxy, formylamino and C₁-C₄-alkylcarbonylamino;
R₈ is hydrogen; C₁-C₄-alkyl; C₁-C₄-alkoxy; F; or Cl; and
X is CH or N,
in free form or in salt form.

2. A process for the preparation of a compound as defined in claim 1 of the formula I, in free form or in salt form, comprising the steps of
a) for the preparation of a compound of the formula I, in which X is N, reaction of a compound of the formula 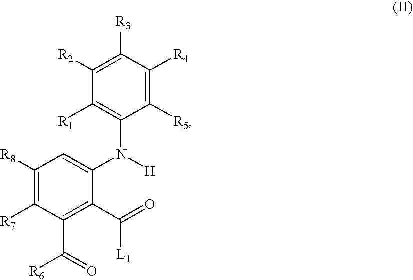
in which R₁, R₂, R₃, R₄, R₅, R₆, R₇ and R₈ are as defined for the formula I and L₁ is a leaving group, in free form or in salt form, with a compound of the formula
H₂N—NH₂ (III),
in free base form or in acid addition salt form, or
b) reaction of a compound of the formula 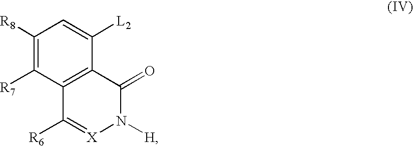
in which R₆, R₇, R₈ and X are as defined for the formula I and L₂ is a leaving group, in free form or in salt form, with a compound of the formula 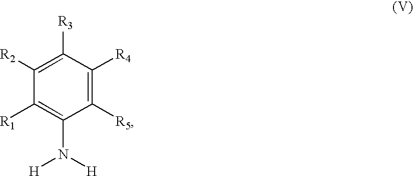
in which R₁, R₂, R₃, R₄ and R₅ are as defined for the formula I, in free form or in salt form, or
c) for the preparation of a compound of the formula I, in which X is CH, R₆ is a group of the formula (R₆ₐ)(R_{6b})(H)C (Ia) and the group of the formula Ia is selected from those groups R₆, which are bonded to the ring carbon atom carrying R₆ via a carbon atom carrying at least one hydrogen atom, intramolecular cyclisation of a compound of the formula 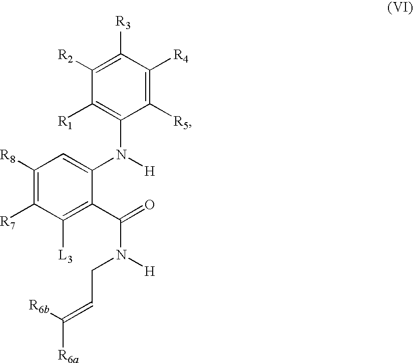
in which R₁, R₂, R₃, R₄, R₅, R₇ and R₈ are as defined for the formula I, Rₕₐ and R_{6b} are as defined for the formula Ia and L₃ is a leaving group, in free form or in salt form,
in each case optionally followed by reduction, oxidation or other functionalisation of the resulting compound and/or by cleavage of any protecting group(s) optionally present, and of recovering the so obtainable compound of the formula I in free form or in salt form.

3. A method for the treatment or prevention of a condition, disease or disorder, in which the modulation of S1P receptors plays a role, comprising administering to a subject in need thereof a therapeutically effective amount of a compound as defined in claim 1 of the formula I, in free form or in pharmaceutically acceptable salt form.

4. A pharmaceutical composition comprising a compound as defined in claim 1 of the formula I, in free form or in pharmaceutically acceptable salt form, as active ingredient, in association with a pharmaceutical carrier or diluent.

5. A compound as defined in claim 1 of the formula I, in free form or in pharmaceutically acceptable salt form, for use as a medicament.

6. A compound as defined in claim 1 of the formula I, in free form or in pharmaceutically acceptable salt form, for the treatment or prevention of a condition, disease or disorder, in which the modulation of S1P receptors plays a role.

7. A combination comprising a therapeutically effective amount of a compound as defined in claim 1 of the formula I, in free form or in pharmaceutically acceptable salt form, and a second drug substance, for simultaneous or sequential administration.

8. The use of a compound as defined in claim 1 of the formula I, in free form or in pharmaceutically acceptable salt form, for the manufacture of a medicament for the treatment or prevention of a condition, disease or disorder, in which the modulation of S1P receptors plays a role.

9. The use of a compound as defined in claim 1 of the formula I, in free form or in pharmaceutically acceptable salt form, as active ingredient in a medicament.

10. The use of a compound as defined in claim 1 of the formula I, in free form or in pharmaceutically acceptable salt form, for the treatment or prevention of a condition, disease or disorder, in which the modulation of S1P receptors plays a role.
